(19) United States
(12) Patent Application Publication
(10) Pub. No.: US 2023/0329528 A1
(43) Pub. Date: Oct. 19, 2023

(54) MITIGATING SMUDGING OF AN ENDOSCOPE WINDOW ELEMENTS DURING A MEDICAL PROCEDURE

(71) Applicant: 270 SURGICAL LTD., Netanya (IL)

(72) Inventor: Avraham LEVY, Kfar Shmaryahu (IL)

(21) Appl. No.: 18/017,709

(22) PCT Filed: Jul. 15, 2021
(86) PCT No.: PCT/IL2021/050866

§ 371 Date: Jan. 24, 2023

Related U.S. Application Data
(60) Provisional application No. 63/057,110, filed on Jul. 27, 2020.

(51) Int. Cl. A61B 1/00; A61B 1/05; A61B 1/06

(52) U.S. Cl. CPC A61B 1/0008; A61B 1/05; A61B 1/0676; A61B 1/00142

(57) ABSTRACT

Provided herein is a distal tip of an endoscope configured to mitigate smudging of window elements at the front face thereof, utilizing various mitigating elements. Further provided are methods of fabricating and using the same in endoscopic procedures.

### TECHNICAL FIELD

The present disclosure relates generally to mitigating smudging off endoscope windows elements (i.e. image/camera window and/or illumination window(s)), during medical procedures.

### BACKGROUND

An endoscope is a medical device used to image an anatomical site (e.g. an anatomical/body cavity, a hollow organ). Unlike some other medical imaging devices, the endoscope is inserted into the anatomical site (e.g. through small incisions made on the skin of the patient). An endoscope can be employed not only to inspect an anatomical site and organs therein (and diagnose a medical condition in the anatomical site) but also as a visual aid in surgical procedures. Medical procedures involving endoscopy include laparoscopy, arthroscopy, cystoscopy, ureterostomy, hysterectomy and other.

During use of the endoscope, i.e., when the endoscope is inserted into a body cavity, smudging and soiling of the endoscope windows elements (camera and/or illumination components) with various bodily fluids, tissues and debris may occur, thereby affecting image quality provided for a user. This may result in affecting the operation of the endoscope by the user and consequently hinder the quality and efficiency of the medical procedure. For example, the endoscope may be removed from the subject body, cleaned and re inserted to continue the medical procedure.

International application publication No. WO 2019/226855 discloses devices, systems and methods for cleaning of elongated instrument surface. US Patent Application No. US 2012/0178995 is directed to method and apparatus for cleaning the field of view of an endoscopic lens.

There is thus a need in the art for apparatuses and methods for mitigating smudging off endoscope windows elements, in particular, for endoscope system having more than one camera.

### SUMMARY

Aspects of the disclosure, according to some embodiments thereof, relate to mitigation of smudging of elements of a distal tip of an endoscope. In some embodiments, aspects of the disclosure relate to an apparatus and method for mitigating smudging of endoscope windows elements, in particular, a front camera/image window and illumination component window(s) of an endoscope having a plurality of cameras at the endoscope distal tip. The disclosed smudging mitigation apparatus allows obtaining an improved image quality, by preventing or at least partially diminishing such smudging during medical procedures. The disclosed apparatus is advantageous as it is cost efficient, customizable, easy to use and does not require a source of energy. The disclosed apparatus provides means to allow displacing various bodily fluids, tissues and debris from the various windows elements, thereby providing clear field of view to the camera and clear path for the illumination component(s) and consequently clearer images and videos to be obtained during surgical operation. Furthermore, by utilizing the apparatus in surgical procedures, the periodic removal of the endoscope for cleaning bodily fluids and debris that smudge the endoscope window elements (camera window and illumination components window(s)), is avoided. In some embodiments, the apparatus is an integral part of the distal end of the endoscope or may be fitted on the distal tip of the endoscope and can reduce the need to repeatedly clean the distal tip, resulting in fewer interruptions, thereby maintaining procedural flow. Consequently, health care providers, such as, surgeons can focus on the surgical procedure and the patient and less on the tools they need to use. The various apparatuses disclosed herein may be disposable or reusable and may advantageously be sterilizable.

According to some embodiments, there is provided an apparatus for mitigating smudging of a camera window and illumination component windows of an endoscope, the apparatus includes a front face exposed to a body cavity and a back face opposite thereto, said back face is configured to fit on a front end of an endoscope distal tip, wherein the apparatus includes one or more apertures configured to fit the camera window, such that a gap is formed between a front face of the camera window and the front face of the apparatus. In some embodiments, the front face of the endoscope further includes ridges, bumps, rims and/or grooves, configured to prevent or divert bodily fluids and/or bodily debris from reaching the camera window and smudging the window.

According to some embodiments, there is provided a method for mitigating smudging of a camera window and illumination component windows positioned on a front end of a distal tip of an endoscope, the method includes fitting and securing an apparatus for mitigating smudging on the front end of the distal tip of the endoscope.

According to some embodiments, there is provided a method for mitigating smudging of a camera window and illumination component windows positioned on a front end of a distal tip of an endoscope, the method includes integrally forming on a distal tip of the endoscope an apparatus for mitigating smudging on the front end of the distal tip of the endoscope.

According to some embodiments, there is provided a smudging mitigation apparatus for mitigating smudging of an endoscope window elements positioned on a front end of a distal tip of the endoscope, the smudging mitigation apparatus includes a front face exposed to a body cavity and a back face opposite thereto, said back face is configured to fit on the front end of the distal tip, wherein the smudging mitigation apparatus further includes one or more apparatus apertures traversing the front face and the back face and configured to correspond to a front lens assembly of a front camera positioned on the front end of the endoscope.

According to some embodiments, the one or more apparatus apertures are further configured to fit corresponding one or more illumination components on the distal tip of the endoscope and provide light to the front camera.

According to some embodiments, the window elements include a front camera window configured to fit within a central apparatus aperture of the one or more apparatus aperture over the front lens assembly of the front camera, and one or more illumination component windows configured to fit within one or more surrounding apparatus aperture of the one or more apparatus aperture over the illumination components.

According to some embodiments, the front end of the distal tip may include one or more apertures configured to fit the one or more apparatus apertures, the front lens assembly and the illumination components.

According to some embodiments, the window elements may be made of sapphire glass.

According to some embodiments, the window elements may be soldered to the respective apertures in the apparatus.

According to some embodiments, the respective apertures are at least partially made of stainless still.

According to some embodiments, the front face may include mitigating elements, comprising ridges, rims, bumps and/or grooves, the mitigating elements are configured to prevent fluids, bodily fluids and/or debris from reaching the window elements.

According to some embodiments, the ridges, rims, bumps and/or grooves may be integrally formed with the front face of the smudging mitigation apparatus. According to some embodiments, the ridges, rims and/or bumps may be attached or mounted to the front face of the smudging mitigation apparatus. According to some embodiments, the ridges, rims, bumps and/or grooves may be made of plastic, metal, rubber, nylon, or any combination thereof.

According to some embodiments, the one or more apparatus apertures and the one or more apertures do not hinder the field of view of the front camera.

According to some embodiments, the diameter of the central apparatus aperture is similar to or larger than the diameter of the front lens assembly.

According to some embodiments, the one or more illumination components is or includes a discrete light source.

According to some embodiments, the diameter of the one or more surrounding apparatus apertures is similar to or larger than the diameter of the corresponding illumination components.

According to some embodiments, the smudging mitigation apparatus is essentially round.

According to some embodiments, a gap is formed between the front end of the distal tip and the back face of the apparatus.

According to some embodiments, the smudging mitigation apparatus may be integrally formed with the front end of a distal tip of the endoscope.

According to some embodiments, the smudging mitigation apparatus includes means for fitting and/or securing to the front end of the endoscope tip.

According to some embodiments, the smudging mitigation apparatus may be reusable. According to some embodiments, the smudging mitigation apparatus may be disposable. According to some embodiments, the smudging mitigation apparatus may be sterilizable.

According to some embodiments, the smudging mitigation is passive.

According to some embodiments, there is provided an endoscope including the smudging mitigation apparatus as disclosed herein. According to some embodiments, the endoscope may include at least two cameras.

According to some embodiments, the at least two cameras include the front camera on the front end of distal tip of the endoscope and a first side-camera. According to some embodiments, the at least two cameras further include a second side-camera, wherein the first side-camera and the second side-camera are positioned on opposite sides of the distal tip of endoscope, and wherein the first side-camera is positioned distally relative to the second side-camera. According to some embodiments, the at least two cameras provide at least about 270 degrees horizontal field-of-view (FOV) of a target area within an anatomical cavity into which the distal tip of the endoscope is inserted.

According to some embodiments, there is provided a method for allowing mitigating smudging of a window element positioned on a distal tip of an endoscope, the method includes:
fitting to a front end of the distal tip of the endoscope a smudging mitigation apparatus having a front face exposed to a body cavity and a back face opposite thereto, said back face is configured to fit on the front end of the distal tip, wherein the smudging mitigation apparatus further includes one or more apparatus apertures traversing the front face and the back face and configured to correspond to a front lens assembly of a front camera positioned on the front end of the endoscope.

According to some embodiments, the method may further include securing the smudging mitigation apparatus to the front end of the distal tip of the endoscope.

According to some embodiments of the method, the front face of the smudging mitigation apparatus comprises mitigating elements comprising ridges, rims, bumps and/or grooves, the mitigating elements are configured to prevent bodily fluids from reaching the window elements.

According to some embodiments, the method may further include a step of forming a gap between the front end of the distal tip and the back face of the apparatus.

According to some embodiments of the method, the mitigation of smudging is passive.

According to some embodiments, there is provided an endoscope distal tip configured for mitigating smudging of the endoscope window elements positioned on a front end of the distal tip of the endoscope, the distal tip includes a front face exposed to a body cavity, said front face includes mitigating elements which include ridges, rims, bumps and/or grooves, said mitigating elements are configured to prevent fluids, bodily fluids and/or debris from reaching the window elements.

According to some embodiments, the mitigating elements are integrally formed with the front face of the distal tip. According to some embodiments, the mitigating elements are attached or mounted to the front face of the tip. According to some embodiments, the mitigating elements may be made of plastic, metal, rubber, nylon, or any combination thereof. Each possibility is a separate embodiment.

According to some embodiments, the window elements may be made of sapphire glass. According to some embodiments, the window elements are soldered to their respective opening in the distal tip. According to some embodiments, the respective opening(s) in the distal tip at least partially comprises stainless still.

According to some embodiments, boundaries formed by the soldering may be configured to further facilitate mitigate smudging.

According to some embodiments, there is provided an endoscope including the distal tip as disclosed herein.

According to some embodiments, there is provided a method of mitigating smudging of window elements at a front end of a distal tip of an endoscope, the method includes forming, applying or mounting mitigating elements on a front face of the front end of the distal tip, the mitigating elements are configured to prevent fluids, bodily fluids and/or debris from reaching the window elements.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1—schematically depict a rigid endoscope, according to some embodiments;

FIG. 2—schematically depicts a medical imaging system including an endoscope having a plurality of cameras which is a specific embodiment of the endoscope of FIG. 1, according to some embodiments;

FIG. 3—shows a perspective view of a front and side camera windows and illumination component windows disposed at a distal tip of an endoscope, according to FIG. 1;

FIG. 4A shows a perspective view of an apparatus for mitigating smudging at the front end of a distal tip of an endoscope, according to FIG. 3;

FIG. 4B shows a perspective view of an apparatus for mitigating smudging at the front end of a distal tip of an endoscope, which is a specific embodiment of the endoscope of FIG. 3; and

FIG. 4C shows a perspective view of an apparatus for mitigating smudging at the front end of a distal tip of an endoscope, which is a specific embodiment of the endoscope of FIG. 3.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

According to some embodiments, there is provided herein an advantageous apparatus for mitigating (preventing or at least partially diminishing) smudging of camera window and/or illumination unit windows (collectively referred to as window elements), positioned on the front end of a distal tip of an endoscope, typically caused by bodily fluids and/or debris. The advantageous apparatus allows an efficient mitigation of smudging by at least partially preventing or diverting the bodily fluids and/or debris from reaching the camera window and/or illumination unit window. In some embodiments, the apparatus is integrally formed with the endoscope tip, i.e., it is an integral part of the tip, whereby, by the structure and/or composition thereof, smudging is mitigated.

As used herein, the term “smudging” relates to soiling, smearing, blurring, blotting or otherwise fouling a surface (such as a window element face) with fluids, bodily fluids, bodily debris and/or bodily tissues.

As used herein, the terms “bodily fluids”, “bodily debris”, “bodily tissues”, relate to various bodily components (such as, blood, interstitial fluid, cells, various tissues, such as fat tissue, and the like), which are found in an internal body region into which the endoscope is inserted, and which may cause smudging of the distal tip of the endoscope (camera window and/or illumination component(s) window(s)).

As used herein, the term “mitigating” relates to at least partially reducing, diminishing, lessening, lowering and/or preventing the smudging of a camera window and/or an illumination component window on the front end of an endoscope distal tip during medical (surgical) procedures.

As used herein, the term “mitigating elements” relates to various patterns that are formed, placed, mounted, attached or otherwise positioned on the front face of the apparatus and/or the distal tip. In some embodiments, such mitigating elements may include, for example, but not limited to: ridges, rims, bumps, grooves, and the like, or any combination thereof, As detailed herein, the mitigating elements may be placed/formed at any desired configuration and may be made of any suitable material. In some embodiments, the mitigating elements may be made of metal, plastic, rubber, silicone, and the like. Each possibility is a separate embodiment.

As used herein, the term “window element” collectively refers to a camera window (for example, a front facing camera) and the respective illumination component window(s).

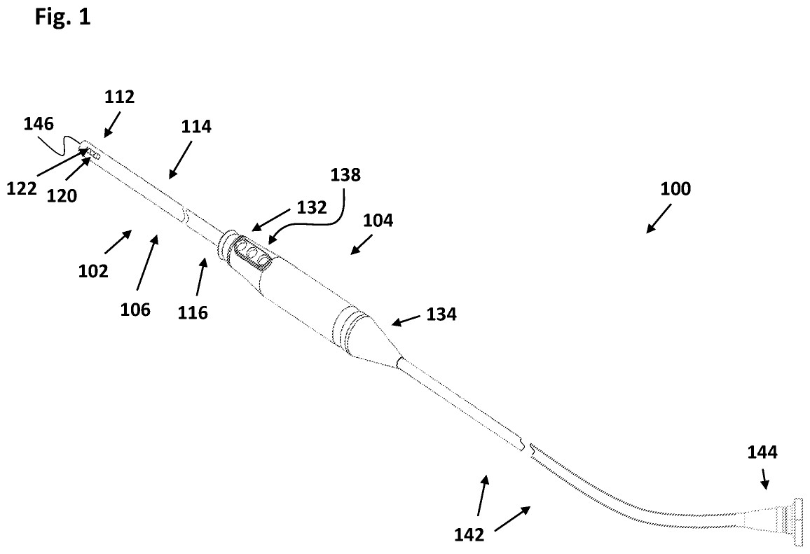

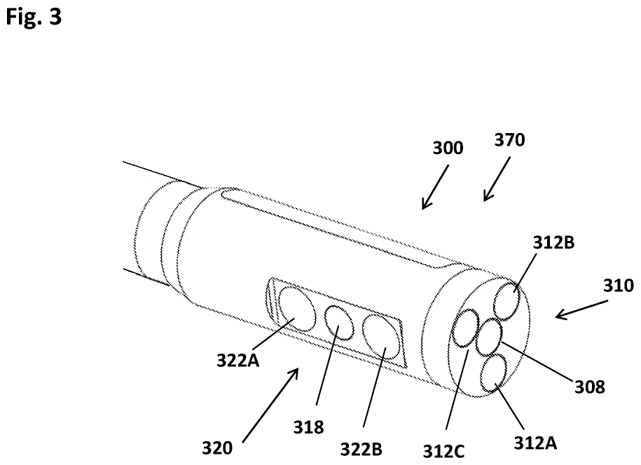

Reference is now made to FIG. 1, which schematically depict a rigid endoscope, according to some embodiments. As shown in FIG. 1, endoscope 100 includes an elongated shaft 102, configured to be inserted into an anatomical site (e.g. an anatomical cavity), and a handle 104, configured to be held by a user (e.g. a surgeon or a robotic arm) of endoscope 100 and to facilitate guiding and manipulation of elongated shaft 102 (particularly a distal section thereof) within the anatomical site. Shaft 102 includes a shaft body 106, e.g. a rigid tubular member. Shaft 102 includes a shaft distal section 112, a shaft central section 114, and a shaft proximal section 116 (i.e. a distal section, a central section, and a proximal section, respectively, of shaft 102). Shaft distal section 112 includes at least two cameras 120 (e.g. a front camera, and at least one side camera, as seen, for example, in FIG. 3) and illumination components 122, such as light emitting diodes (LEDs). According to some embodiments, each of the illumination components includes one or more light-emitting diodes (LEDs). According to some embodiments, each of illumination components 122 is or includes a discrete light source. According to some embodiments, wherein illumination components 122 include LEDs, the LEDs may include, for example, one or more white light LEDs, infrared LEDs, near infrared LEDs, an ultraviolet LED, and/or a combination thereof. It is noted that in embodiments wherein illumination components include LEDs configured to produce light outside the visible spectrum (e.g. an infrared spectrum, a UV spectrum), cameras 120 may include suitable sensors configured to detect such type of light (e.g. infrared light, ultraviolet). That is, cameras 120 will have capacities of e.g. infrared cameras and so on. According to some embodiments, the illumination components may include the distal tips of respective optical fibers (not shown).

The handle 104 may include a user control interface 138 configured to allow a user to control endoscope 100 functions. User control interface 138 may be functionally associated with cameras 120 and illumination components 122 via an electronic coupling between shaft 102 and handle 104. According to some embodiments, user control interface 138 may allow, for example, to control zoom, focus, multifocal views, record/stop recording, freeze frame functions, etc., of cameras 120 and/or to adjust the light intensity provided by illumination components 122.

Each of cameras 120 may include a sensor, such as a charge coupled device (CCD) image sensor or a complementary metal oxide semiconductor (CMOS) image sensor, and a camera lens (e.g. an extreme wide-angle lens) or a lens assembly. Cameras 120 may be configured to provide a continuous/panoramic/surround field-of-view (FOV), as elaborated on below.

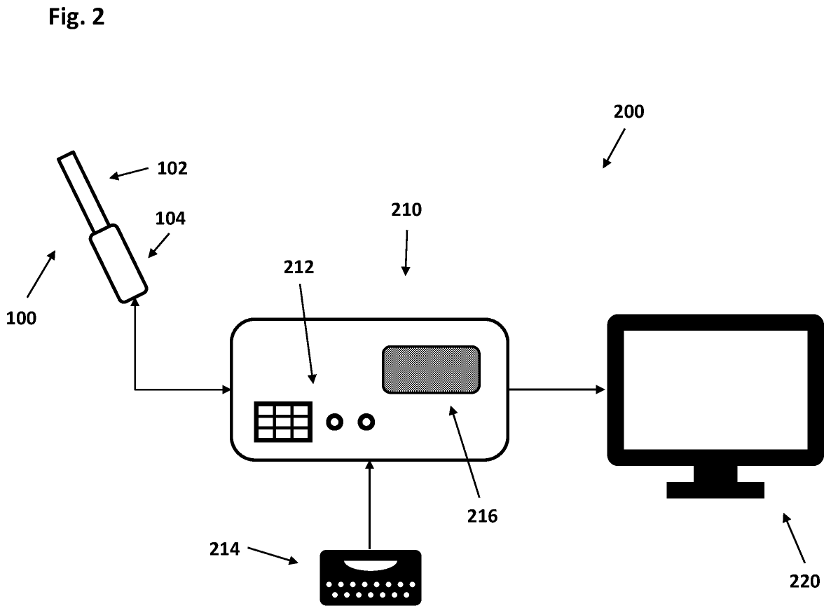

Reference is now made to FIG. 2, which schematically depicts a medical imaging system 200, according to some embodiments. Medical imaging system 200 includes an endoscope 100 having a plurality of cameras which is a specific embodiment of the endoscope of FIG. 1, a main control unit 210, and a monitor 220.

As shown in FIG. 2, Endoscope 100 and monitor 220 may each be functionally associated with main control unit 210. Main control unit 210 includes processing circuitry (e.g. one or more processors and memory components) configured to process data from at least two cameras 120 (not shown in FIG. 2 but depicted in FIG. 1 at a shaft distal section 112), such as to display the captured image, and video streams on monitor 220. In particular, the processing circuitry may be configured to process the data received from each of the at least two cameras 120, such as to produce therefrom video files/streams providing a 3D, panoramic/surround view of the anatomical site. According to some embodiments, the processing circuitry may be configured to process the data received from cameras 120 to produce a combined video stream providing a continuous and consistent (seamless) panoramic view of the anatomical site.

Main control unit 210 may include a user interface 212 (e.g. buttons and/or knobs, a touch panel, a touch screen) configured to allow a user to operate main control unit 210 and/or may allow control thereof using one or more input devices 214, e.g. an external user control interface connectable thereto such as a keyboard, a mouse, a portable computer, and/or even a mobile computational device e.g. a smartphone or a tablet. According to some embodiments, input devices 214 may include a voice controller. According to some embodiments, main control unit 210 may further be configured to partially or even fully operate at least two cameras 120 and illumination components 122 (shown in FIG. 1 at a shaft distal section 112). Some operational aspects may be operated automatically, for example, according to some embodiments, the supply of power to endoscope 100 components, such as at least two cameras 120 and illumination components 122, while other operational aspects or functions may be operated using user interface 212 and/or input devices 214. According to some embodiments, main control unit 210 may include a display 216 (for example, the touch screen and/or another screen) for presenting information regarding the operation of endoscope 100, such as the brightness levels of at least two cameras 120, zoom options, focus, and the like. According to some embodiments, wherein display 216 is a touch screen, display 216 may further allow controlling for example, the zoom, focus, multifocal imaging, selecting images from specific lens assemblies, compiling images from various lens assemblies, creating a multifocal image, record/stop recording functions, freeze frame function, and/or the brightness of at least two cameras 120, and/or to adjust the light intensity of illumination components 122. According to some embodiments, the choice of information presented may be controlled using user interface 212, user control interface 138, and/or input devices 214.

According to some embodiments, endoscope 100 is functionally associated with main control unit 210 via a utility cable 142 (shown in FIG. 1), connected to or configured to be connected to handle proximal section 134, and further configured to be connected to main control unit 210 (via, for example, a plug 144 or a port). Utility cable 142 may include at least one data cable for receiving video signals from at least two cameras 120, and at least one power cable for providing electrical power to at least two cameras 120 and to illumination components 122, as well as to operationally control parameters of at least two cameras 120 and illumination components 122, such as the light intensity. Additionally or alternatively, according to some embodiments, endoscope 100 may include a wireless communication unit (e.g. a Bluetooth antenna or Wi-Fi) configured to communicatively associate endoscope 100 with main control unit 210. According to some embodiments, endoscope 100 is configured to be powered by a replaceable and/or rechargeable battery included therein, i.e. inside handle 104. According to some embodiments, wherein illumination components 122 include distal tips of optical fibers and wherein the light source(s) is positioned in main control unit 210, cable 142 will also include one or more optical fibers configured to guide the light produced by the light source(s) to an optical fiber(s) in handle 104, wherefrom the light will be guided to optical fibers in shaft 102.

Monitor 220 is configured to display images and, in particular, to display multifocal stream videos captured by at least two cameras 120, and may be connected to main control unit 210 by a cable (e.g. a video cable) or wirelessly. According to some embodiments, monitor 220 may be configured to display thereon information regarding the operation of endoscope 100, as specified above. According to some embodiments, monitor 220, or a part thereof, may function as a touch screen. According to some such embodiments, the touch screen may be used to operate main control unit 210. According to some embodiments, images/videos from different cameras (from at least two cameras 120) or from different lens assemblies of the different cameras, may be displayed separately (e.g. side-by-side, picture on picture, in an equal aspect ratio, in an un-equal aspect ratio, in multiple copies of one or more of the video streams, and the like) on monitor 220, and/or may be presented as a single panoramic/surround, optionally multifocal or 3D image/video. According to some embodiments, user interface 212 and/or input devices 214 and/or user control interface 138 are configured to allow switching between images/videos corresponding to different field of views (of different cameras) and/or of different field of views (obtained from different lens assemblies of one or more sensors). For example, according to some embodiments, wherein at least two cameras 120 include a front camera 120a, a first side camera 120b, and a second side cameras 120c: switching between footage(s) captured by one or more lens assemblies from front camera 120a to footage(s) captured by one or more lens assemblies of first side camera 120b, switching between footage(s) captured by one or more lens assemblies of front camera 120a to footage(s) captured by one or more lens assemblies of second side cameras 120c, or switching between panoramic/surround video(s) generated from the footage(s) of all of cameras 120a, 120b, and 120c to footage captured by one of cameras 120a, 120b, or 120c. Cameras 120a, 120b, and 120c are depicted together in FIG. 2. According to some embodiments, main control unit 210 may be associated with a plurality of monitors, such as monitor 220, thereby allowing displaying different videos and images on each. For example, main control unit 210 may be associated with four monitors, such as to allow displaying videos from each of cameras 120a, 120b, 120c on three of the monitors, respectively, and a panoramic video (corresponding to the combination of the three videos) on the fourth monitor, which may be wider than the other three.

The field-of-view (FOV) provided by endoscope 100 is the combination of the respective FOVs provided by each of the at least two cameras 120. At least two cameras 120 may be configured to provide a continuous and consistent FOV, or at least a continuous and consistent horizontal FOV (HFOV).

According to some embodiments, each of the at least two cameras 120 is associated with a respective illumination component from illumination components 122, which is configured to illuminate the FOV of the camera. Thus, according to some embodiments, a front camera may be associated with a respective front illumination component, a first side-camera may be associated with a respective first side-illumination component, and a second side-camera may be associated with a respective second side-illumination component.

According to some embodiments, cameras 120 may include only two cameras, both of which are side cameras with fisheye lenses. According to some embodiments, cameras 120 include only two cameras: a front camera and a side camera.

According to some embodiments, the illumination components may include one or more illumination components. In some embodiments, the illumination components may include at least one illumination component(s). According to some embodiments, the illumination components may include two or more illumination components. In some embodiments, the illumination components may include at least two illumination components. According to some embodiments, the illumination components may include three or more illumination components. In some embodiments, each camera is associated with corresponding one or more illumination components. In some embodiments, each camera is associated with corresponding at least one illumination component(s).

According to some embodiments, endoscope 100 may be (i) directly maneuvered by a user through the manipulation of handle 104, as well as (ii) indirectly maneuvered, via robotics, e.g. using a robotic arm or other suitable gripping means configured to allow manipulation of handle 104.

Reference is now made to FIG. 3, which depicts a perspective close up view of a front and side cameras at a tip of an endoscope, according to FIG. 1. As shown in FIG. 3, a distal tip 370 of an endoscope 300, includes a front camera window 308, which faces a front view 310 of distal tip 370, and a side camera window 318 which faces a side view 320 of distal tip 370. A front camera may be positioned behind (i.e. proximally to) front camera window 308 (and is, thus, hidden from view in FIG. 3). Adjacent to front camera window 308 located front illumination components windows configured to provide light/illumination to the front camera (not shown) field of view (not shown). The front illumination components windows are shown as illumination windows 312A, 312B and 312C. The illumination source/module of a front illumination unit (component) may be positioned behind the front illumination components windows 312A, 312B and 312C, respectively (and are, thus, not visible in FIG. 3). According to some embodiments, front camera window 308 and front illumination components windows 312 may be referred to collectively as window(s) elements.

As further shown in FIG. 3, a side camera element may be positioned behind (i.e. proximally to) side camera window 318 (and is, thus, hidden from view in FIG. 3). Adjacent to side camera window 318 are side illumination components windows 322A and 322B, configured to provide light to the side camera field of view (not shown). The illumination sources/modules of the side illumination unit/component may be positioned behind the side illumination components windows 322A and 322B, respectively (and are, thus, not visible in FIG. 3). According to some embodiments, side camera window 318 and side illumination components windows 322 may be referred to collectively as side window elements.

According to some embodiments, each of the illumination modules may include a plurality of LEDs, for example, two, three, or four LEDs which may be arranged in an array. According to some embodiments, the LEDs may emit light at the same wavelength. According to some alternative embodiments, different LEDs may emit light at different wavelengths, respectively.

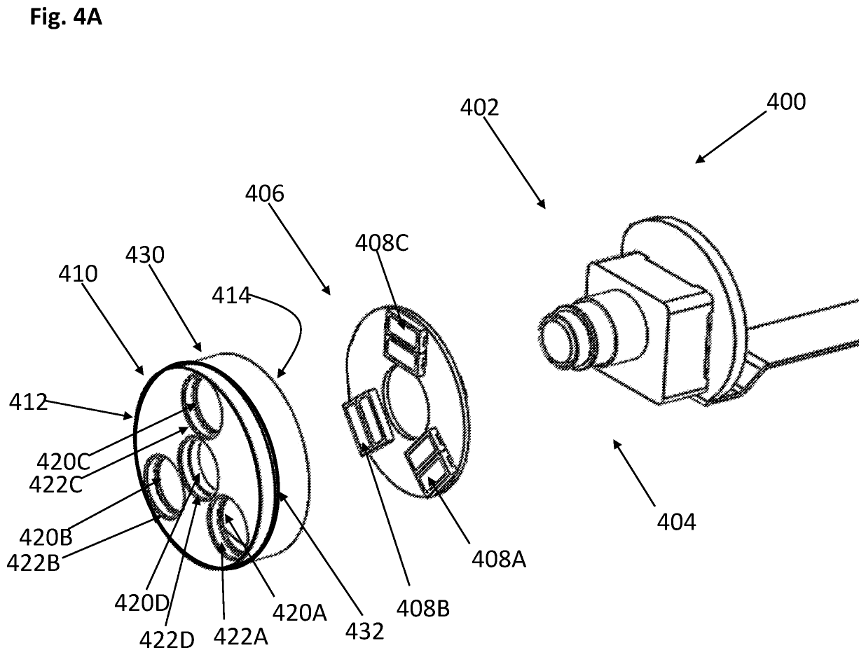

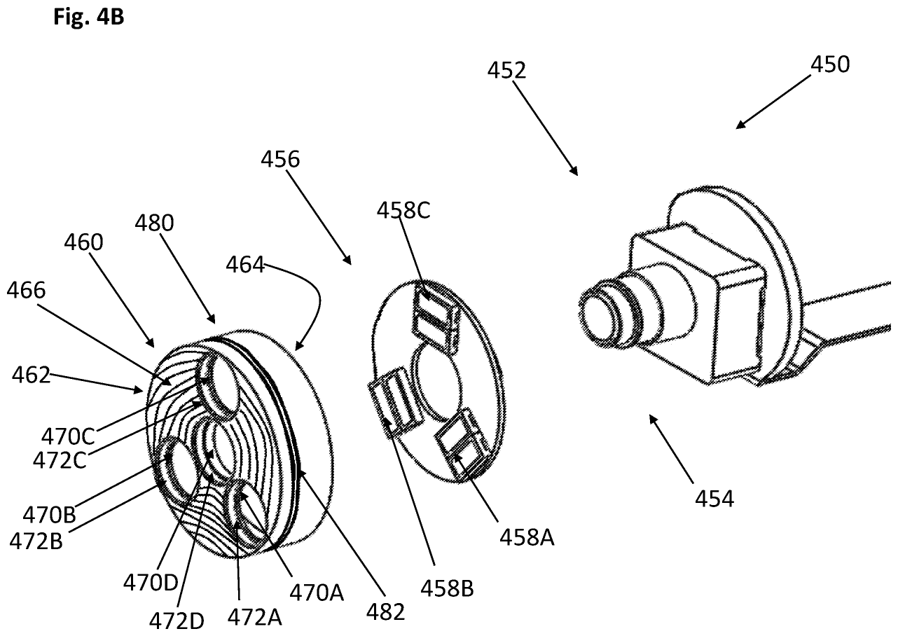

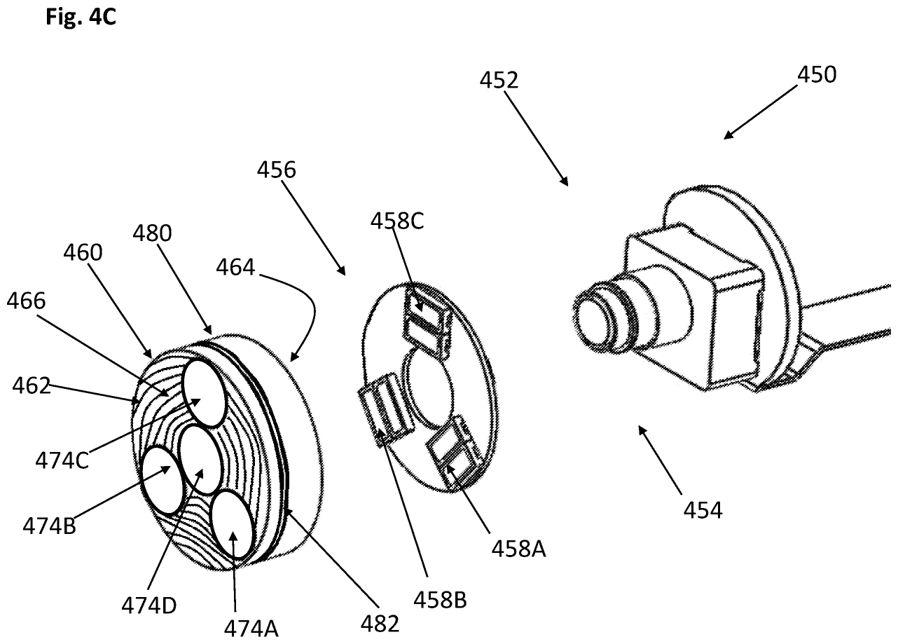

Reference is now made to FIGS. 4A-C which show views of an apparatus for mitigating smudging off a distal tip of an endoscope such as endoscope 300 of FIG. 3. FIG. 4A shows a perspective view of a front camera 402, and front lens assembly 404 of the front camera 402, wherein front camera 402 is configured to be positioned on a front end 430 of distal tip 400 of an endoscope (not shown for simplicity). Front camera 402 may include a sensor (not shown), such as a charge coupled device (CCD) image sensor or a complementary metal oxide semiconductor (CMOS) image sensor, in addition to the front lens assembly 404 (which may be, for example, an extreme wide-angle lens assembly). Further shown is front illumination unit 406 which includes illumination components, shown as illumination components/modules 408A-C. Each of the illumination components, such as illumination components 408A-C is or includes a discrete light source. For example, one or more of the illumination components, may include a light emitting diodes (LEDs). According to some embodiments, each of the illumination components includes one or more light-emitting diodes (LEDs). According to some embodiments, the LEDs may include, for example, one or more white light LEDs, infrared LEDs, near infrared LEDs, an ultraviolet LED, and/or a combination thereof. According to some embodiments, front illumination unit 406 may include one, two, three or more illumination modules. According to some embodiments, each of the illumination modules includes one or more light-emitting diodes (LEDs). Further shown is a smudging mitigation apparatus 410. As shown in FIG. 4A, smudging mitigation apparatus 410 includes a front face 412 and a back face 414. Front face 412 is configured to face internal body cavity into which distal tip 400 of the endoscope is inserted. Back face 414 faces the front end 430 of the endoscope. As further shown in FIG. 4A, front end 430 of distal tip 400 includes apertures 420A-D which correspond in shape, size (for example, diameter) and/or location/position to front lens assembly 404 of front lens assembly 404 and/or illumination components 408. For example, aperture 420D corresponds to front lens assembly 404, and apertures 420A-C correspond to illumination components/modules 408A-C. Thus, when front end 430 is placed/fitted over the front lens assembly 404 and/or illumination components 408A-C they are correctly placed/fitted, such that none of the apertures 420A-D hinder or cover the faces of the front lens assembly 404 and/or illumination components 408A-C. Furthermore, the smudging mitigation apparatus 410 includes one or more apparatus apertures, shown as apparatus apertures 422A-D traversing the front face 412 and back face 414, wherein the apparatus apertures 422A-D correspond to apertures 420A-D, and to the front lens assembly 404 and/or illumination components 408 on the front end 430 of distal tip 400. Each of the apparatus apertures 422A-D correspond in size (for example, diameter) and/or location to corresponding apertures 420A-D and to component on the front end 430 on the distal tip 400 of the endoscope. For example, apparatus aperture 422D corresponds to aperture 420D and to front lens assembly 404, and apparatus apertures 422A-C correspond to apertures 420A-C and to illumination components/modules 408A-C. Thus, when the smudging mitigation apparatus 410 is placed/fitted over the front end 430 of the endoscope, the lens assembly 404 and/or illumination components 408A-C are correctly placed/fitted, such that none of the apparatus apertures 422A-D hinder or cover the faces of front lens assembly 404 and/or illumination components 408A-C. In other words, after the smudging mitigation apparatus 410 is placed over the front end 430 of the endoscope, the front camera 402 maintains its full field of view and the illumination components 408 can provide illumination without obstruction. In some embodiments, each of the apertures 420 and of the apparatus apertures 422 diameter is equal to or larger than the corresponding diameter of the front lens assembly 404 and/or illumination components 408. In some embodiments, after the smudging mitigation apparatus 410 is fitted over the front end 430, the smudging mitigation apparatus 410 may be secured thereto, by any suitable means, such as, hinges, snaps, dedicated screws, wires, bands, by being screwed, and the like. In some embodiments, after the smudging mitigation apparatus 410 is fitted over front end 430 of distal tip 400, a gap 432 may be formed between a front face of front end 430 and the back face 414 of the smudging mitigation apparatus 410. The gap 432 may be in the range of about 0.001-2 mm, or any subranges thereof. For example, the gap may be in the range of about 0.001-0.01 mm. For example, the gap may be in the range of about 0.01-0.1 mm. For example, the gap may be in the range of about 0.1-1. For example, the gap may be in the range of about 0.1-2 mm. The gap 432 thus formed, aids in preventing and/or diverting the bodily fluids/tissues/debris from reaching endoscope window elements such as a front camera window and the illumination component(s) windows, for example front camera window 308 and front illumination component windows 312A-C of FIG. 3, to thereby prevent or at least partially diminish smudging thereof.

Reference is now made to FIG. 4B, which shows a perspective view of a front camera 452, and a front lens assembly 454 of front camera 452, wherein the front camera 452 is configured to be positioned on a front end 480 of a distal tip 450 of an endoscope (not shown for simplicity). Front camera 452 may include a sensor (not shown), such as a charge coupled device (CCD) image sensor or a complementary metal oxide semiconductor (CMOS) image sensor, in addition to the front lens assembly 454 (which may be, for example, an extreme wide-angle lens assembly). Further shown is front illumination unit 456 which includes illumination components, shown as illumination components/modules 458A-C. Illumination components, such as illumination components 458A-C are or include a discrete light source. For example, one or more of the illumination components may include a light emitting diodes (LEDs). According to some embodiments, the LEDs may include, for example, one or more white light LEDs, infrared LEDs, near infrared LEDs, an ultraviolet LED, and/or a combination thereof. According to some embodiments, front illumination unit 456 may include one, two, three or more illumination modules. According to some embodiments, each of the illumination modules includes one or more light-emitting diodes (LEDs). Further shown is smudging mitigation apparatus 460. As shown in FIG. 4B, smudging mitigation apparatus 460 includes a front face 462 and a back face 464. Front face 462 is configured to face internal body cavity into which distal tip 450 of the endoscope is inserted. Back face 464 faces the front end 480 of the endoscope. As further shown in FIG. 4B, front end 480 of distal tip 450 includes apertures 470A-D which correspond in shape, size (for example, diameter) and/or location/position to front lens assembly 454 of front camera 452 and/or illumination components 458. For example, aperture 470D corresponds to front lens assembly 454, and apertures 470A-C correspond to illumination components/modules 458A-C. Thus, when front end 480 is placed/fitted over the front lens assembly 454 and/or illumination components 458A-C they are correctly placed/fitted, such that none of the apertures 470A-D hinder or cover the faces of the front lens assembly 454 and/or illumination components 458A-C. Furthermore, the smudging mitigation apparatus 460 includes apparatus apertures 472A-D traversing the front face 462 and back face 464, wherein the apparatus apertures 472A-D correspond to apertures 470A-D, and to the front lens assembly 454 and/or illumination components 458 on the front end 480 of distal tip 450. Each of the apparatus apertures 472A-D may correspond in shape, size (for example, diameter) and/or location/position to a corresponding apertures 470A-D and to component on the front end 480 on the distal tip 450 of the endoscope. For example, apparatus aperture 472D corresponds to aperture 470D and to front lens assembly 454, and apparatus apertures 472A-C correspond to illumination components/modules 458A-C. Thus, when the smudging mitigation apparatus 460 is placed/fitted over the front end 480 of the endoscope, the front lens assembly 454 and/or illumination components 458A-C are correctly placed/fitted, such that none of the apparatus aperture 472A-D hinder or cover the faces of front lens assembly 454 and/or illumination component 458A-C. As shown in FIG. 4B, the front face 462 may further include a pattern of ridges, rims, bumps and/or grooves, shown as patterns 466. The pattern 466 on the front face 462 may have any geometrical shape or pattern, including, for example, straight lines, circular lines, curved lines, and the like, or any combination thereof. In some embodiments, the patterns 466 (i.e., grooves, ridges, rims, bumps, and the like) may be integral part of the front face 462 and/or may be added/placed thereto. For example, the patterns 466 may be integrally formed on the front face 462. For example, the patterns 466 may be fixed/placed on the front face 462, permanently or transiently, for example by being mounted thereon, attached thereto, adhered on the front face, and the like. In some embodiments, the patterns 466 on the front face 462 further aid in preventing and/or diverting the bodily fluids, tissues and/or debris, from reaching, touching or contacting endoscope window elements such as a front camera window and an illumination component windows as illustrated in relation to FIG. 4C, below. Thus, in some embodiments, after the smudging mitigation apparatus 460 is placed over the front end 480 of the endoscope, the front camera 452 maintains its full field of view and the illumination components 458 can provide illumination without any obstruction. In some embodiments, each of the aperture 470 and of the apparatus apertures 472 diameter is equal to or larger than the corresponding diameter of the front lens assembly 454 and/or illumination component 458. In some embodiments, after the smudging mitigation apparatus 460 is fitted over the front end 480, the smudging mitigation apparatus 460 may be secured thereto, by any suitable means, such as, hinges, snaps, dedicated screws, wires, bands, by being screwed, and the like. In some embodiments, after the smudging mitigation apparatus 460 is fitted over the front end 480 of distal tip 450, a gap 482 may be formed between the front end 480 and the back face 464 of the smudging mitigation apparatus 460. For example, the gap 482 may be in the range of about 0.001-2 millimeters. For example, the gap 482 may be in the range of about 0.001-0.01 millimeters. For example, the gap 482 may be in the range of about 0.01-0.1 millimeters. For example, the gap 482 may be in the range of about 0.1-1. For example, the gap 482 may be in the range of about 0.1-2 millimeters. The gap 482 thus formed, in addition to the front face pattern 466, aids in preventing and/or diverting the bodily fluids/tissues/debris from reaching the camera window and/or illumination component window surfaces and thereby prevents or at least partially diminish smudging thereof.

Reference is now made to FIG. 4C, which illustrates smudging mitigation apparatus 460 which includes front window elements 474. Front window elements 474 may include: a central window 474D (camera window), and three surrounding windows 474A, 474B, and 474C (illumination component windows), which surround central window 474D. Front camera 452 may be positioned behind (i.e. proximally to) central window 474D, such that central window 474D may also be referred to as front camera window 474D. Three illumination components/modules 458A, 458B and 458C of front illumination unit 456 may be positioned behind the three windows 474A, 474B, and 474C, respectively, which may also be referred to herein as illumination component windows 474A, 474B, and 474C. According to some embodiments, front window elements 474A-D may differ from one another in shape and/or in size. In particular, according to some embodiments, front camera window 474D may differ in shape and/or size from illumination component windows 474A, 474B, and 474C.

Each of the apparatus apertures 422, 472 (shown in FIGS. 4A-B) has set therein a respective window 474 (e.g. a windowpane). For example, apparatus aperture 422D, 472D, corresponds to front lens assembly 404, 454, and apparatus apertures 422A-C correspond to illumination components/modules 408A-C. Each of the windows of front window elements 474 is set within a corresponding apparatus aperture from the smudging mitigating apparatus 410, 460. For example, front camera window 474D is set within apparatus aperture 422D, 472D. More specifically, each of the apertures is shaped and dimensioned to have affixed therein (for example, by soldering) a corresponding window.

In some embodiments, the smudging mitigation apparatus is adjustable, in particular, by designing the apertures to fit in size and/or position to the corresponding lens assembly and/or illumination components, such that smudging thereof is mitigated on the one hand, while functionality (for example, field of view, depth of field, illumination intensity, etc.) is not affected nor hindered.

In some embodiments, the smudging mitigation apparatus may be disposable, such that it is fitted to the distal tip the endoscope prior to use (i.e, prior to being inserted into the subject body). In some embodiments, the smudging mitigation apparatus may be reusable.

In some embodiments, the smudging mitigation apparatus allows passive smudging mitigation. In some embodiments, the smudging mitigation apparatus is configured to mitigate smudging in a passive manner, i.e., without use of electrical power and/or the maneuvering of moving parts.

In some embodiments, the smudging mitigation apparatus may be permanently secured to the distal tip of the endoscope. In some embodiments, the smudging mitigation apparatus may be removable from the distal tip of the endoscope.

In some embodiments, the smudging mitigation apparatus may be integrally formed with the distal tip of the endoscope.

In some embodiments, the smudging mitigation apparatus may be constructed from any biocompatible material, including, for example, but not limited to: metal, plastic, rubber, silicone, and the like, or any combinations thereof. Each possibility is a separate embodiment.

In some embodiments, the smudging mitigation apparatus may be sterilizable (for example, by autoclave).

In some embodiments, the mitigating elements (patterns) on the front face of the smudging mitigation apparatus may be an integral part of the front face. In some embodiments, the patterns on the front face of the smudging mitigation apparatus may be integrally formed with the front face.

In some embodiments, the patterns on the front face of the smudging mitigation apparatus may be mounted on or positioned on the front face. For example, the patterns may be fixed to, adhered to, attached to, soldered to the front face.

In some embodiments, the patterns, such as, ridges, lines, rims, bumps, and the like, may be made of the same material as the front face, or may be made from a different material. In some embodiments, the patterns may be made of metal, plastic, rubber, silicone, and the like. Each possibility is a separate embodiment.

In some embodiments, the patterns on the front face may have any geometrical shape/form/design, including, for example but not limited to: straight lines, circular lines, curved lines, and the like, or any combination thereof. Each possibility is a separate embodiment.

In some embodiments, the patterns may be selected from grooves and/or ridges on the front face.

In some embodiments, the patterns are configured to at least partially divert or prevent bodily fluids, bodily debris and/or bodily tissues from reaching or touching the camera window and/or the illumination components windows (collectively, the window elements), to thereby mitigate smudging thereof.

In some embodiments, the patterns may include, for example, but not limited to: a pattern of ridges, rims, bumps, grooves, and the like, or any combination thereof. In some embodiments, the pattern may have any geometrical shape or pattern, including, for example, straight lines, circular lines, curved lines, and the like, or any combination thereof. In some embodiments, the patterns may be integral part of the front face of the endoscope and/or may be added/placed thereto. For example, the patterns may be integrally formed on the front face. For example, the patterns may be fixed/placed on the front face, permanently or transiently, for example by being mounted thereon, attached thereto, adhered on the front face, soldered thereto, and the like. In some embodiments, the patterns may aid in preventing and/or diverting the bodily fluids, tissues and/or debris, from reaching, touching or contacting endoscope window elements such as a front camera window and an illumination component windows. In some embodiments, the patterns serve as passive mitigation means.

According to some embodiments, the front face may be made of any suitable material, or combination of materials, including, for example, stainless still and/or sapphire, which may protect and shield the cameras and/or the light sources. In some embodiments, the window elements of the front face (i.e., those covering the camera and/or the light sources) may be made of sapphire glass, which are soldered (for example, using a noble-metal (such as, gold and/or platinum) as solder material), within their respective holes/apertures/opening on the front face.

According to some embodiments, the regions of soldering (i.e., between the glass element and the stainless still elements of the front face) may further aid in smudging mitigation of the front camera and/or illumination components, in addition to or alternatively to the patterns on the front face. In some embodiments, without wishing to be bound to any theory or mechanism, the boundaries formed between two different materials (i.e., at the points/regions of soldering) reduce smudging, as they prevent or at least partially reduce the flow of debris or other bodily fluids over the front camera and/or the front illumination sources. Thus, according to some embodiments, passive smudging mitigation is facilitated by the boundaries formed between at least two different materials at the front face of the endoscope. In some exemplary embodiments, the boundaries are formed by soldering material located at the regions of contact between glass elements (for example, windows of the camera and/or illumination components) and the front face body/housing (made, for example, from still).

According to further embodiments, the relative size/diameter (i.e., ratio) of the camera compared to the diameter of the front face may further aid or participate in smudging mitigation of the front camera in addition to or alternatively to the mitigating elements on the front face and/or the soldering regions. According to some embodiments, the relative size (diameter) of the front camera relative to the front face of the endoscope tip may be in the range of about 5-15% (for example, 7-10%) as the size (diameter) of the front face. Accordingly, such relatively small ratio may further facilitate the passive mitigation of smudging of the front camera, as less debris or other soiling are to come in contact with the front camera.

According to some embodiments, there is provided an endoscope distal tip configured for mitigating smudging of endoscope window elements positioned on the front end of the distal tip of the endoscope, the distal tip includes a front face exposed to a body cavity, the front face includes mitigating elements, which may include, for example, ridges, rims, bumps and/or grooves, wherein the mitigating elements are configured to prevent fluids, bodily fluids and/or debris from reaching the window elements.

In some embodiments, the mitigating elements are integrally formed with the front face of the distal tip. In some embodiments the mitigating elements are attached or mounted to the front face of the distal tip.

In some embodiments, the window elements may be made of sapphire glass. In some embodiments, the window elements may be soldered to their respective opening in the distal tip. In some embodiments, the respective opening in the distal tip at least partially includes stainless still. In some embodiments, the rim of the opening includes stainless still.

According to some embodiments, boundaries formed by the soldering are configured to further mitigate smudging.

According to some embodiments, there is provided a method of mitigating smudging of window elements at a front end of a distal tip of an endoscope, the method includes forming, applying or mounting mitigating elements on a front face of the front end of the distal tip, wherein the mitigating elements are configured to prevent fluids, bodily fluids and/or debris from reaching the window elements.

According to some embodiments, there is provided a smudging mitigation apparatus for mitigating smudging of an endoscope window elements positioned on a front end of a distal tip of the endoscope, the smudging mitigation apparatus includes a front face exposed to a body cavity and a back face opposite thereto, said back face is configured to fit on a front end of the distal tip, wherein the smudging mitigation apparatus comprises one or more apparatus apertures configured to correspond a front lens assembly of a front camera positioned on the distal tip of the endoscope. In some embodiments, a gap is formed between a front end of the distal tip and the back face of the apparatus.

According to some embodiments, there is provided an apparatus for mitigating smudging of a camera window positioned on a front end of a distal tip of an endoscope, the apparatus includes a front face exposed to a body cavity and a back face opposite thereto, the back face is configured to fit on a front end of the distal tip, wherein the front face and the back face of the apparatus include one or more apertures configured to fit at least the front lens assembly, such that a gap, is formed between the front end and the back face of the apparatus. In some embodiments, the gap is in the range of about 0.001-2 millimeters. In some embodiments, the gap is in the range of about 0.001-0.01 millimeters. In some embodiments, the gap is in the range of about 0.01-0.1 millimeters. In some embodiments, the gap is in the range of about 0.1-1, 0.1-2 millimeters.

In some embodiments, the front face may include patterns (mitigating elements), such as, ridges, rims, bumps and/or grooves configured to prevent bodily fluids and/or debris from reaching the window elements. Each possibility is a separate embodiment.

In some embodiments the mitigating elements (such as, ridges, rims, bumps and/or grooves) may be integrally formed with the front face of the smudging mitigation apparatus. In some embodiments, the ridges, rims and/or bumps may be attached on or mounted to the front face of the smudging mitigation apparatus. In some embodiments, the ridges, rims, bumps and/or grooves may be made of plastic, metal, rubber, nylon, silicone or any combination thereof.

In some embodiments, the apertures of the smudging mitigation apparatus do not hinder the field of view of the camera. In some embodiments, the diameter of the aperture which corresponds to front lens assembly is similar to or larger than the diameter of the front lens assembly. In some embodiments, the apertures are further configured to fit one or more corresponding illumination components on the distal end of the endoscope. In some embodiments, the diameter of the apertures is similar to or larger than the diameter of the corresponding illumination components. In some embodiments, the apparatus includes at the front face thereof boundaries formed (for example, by soldering) between glass components thereof and the body/housing thereof. In some embodiments, the boundaries are formed by soldering material (such as, gold or platinum) between sapphire glass elements (i.e., those covering the front camera and/or illumination components), and their respective openings/apertures in the front face body/housing, which may be made of, for example, stainless still.

In some embodiments, smudging mitigation apparatus is essentially round or having a disc-like shape. In some embodiments, the diameter of the smudging mitigation apparatus corresponds to the diameter of the distal tip of the endoscope.

In some embodiments the smudging mitigation apparatus may further include means for fitting and/or securing to the front end of the distal tip of the endoscope. In some embodiments, the means may include, snaps, screws, screwing means, belts, cables, and the like, or any combination thereof. Each possibility is a separate embodiment.

According to some embodiments, there is provided an endoscope having the smudging mitigation apparatus as disclosed herein on the distal tip thereof.

According to some embodiments, there is provided a method for allowing mitigating smudging of a camera window and/or illumination components windows positioned at the front end of a distal tip of an endoscope, the method includes fitting/placing/securing a mitigating smudging apparatus as disclosed herein to the distal tip of the endoscope, prior to being inserted into a body of a subject.

According to some embodiments, there is provided a smudging mitigation apparatus for mitigating smudging of an endoscope window elements positioned on a front end of a distal tip of the endoscope, the smudging mitigation apparatus includes a front face exposed to a body cavity and a back face opposite thereto, said back face is configured to fit on a front end of the distal tip, wherein the smudging mitigation apparatus further includes one or more apparatus apertures traversing the front face and the back face and configured to correspond to a front lens assembly of a front camera positioned on the front end of the endoscope, such that a gap is formed between the front end of the distal tip and the back face of the apparatus.

According to some embodiments, there is provided a method for allowing mitigating smudging of a window element positioned on a distal tip of an endoscope, the method includes fitting to the front end of the distal tip of the endoscope a smudging mitigation apparatus having a front face exposed to a body cavity and a back face opposite thereto, said back face is configured to fit on a front end of the distal tip, wherein the smudging mitigation apparatus further includes one or more apparatus apertures traversing the front face and the back face and configured to correspond to a front lens assembly of a front camera positioned on the front end of the endoscope, such that a gap is formed between the front end of the distal tip and the back face of the apparatus.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-50. (canceled)

51. A smudging mitigation apparatus for mitigating smudging of an endoscope window elements positioned on a front end of a distal tip of the endoscope, the smudging mitigation apparatus comprising a front face exposed to a body cavity and a back face opposite thereto, said back face configured to fit on the front end of the distal tip,
wherein the smudging mitigation apparatus further comprises one or more apparatus apertures traversing the front face and the back face and configured to correspond to a front lens assembly of a front camera positioned on the front end of the endoscope, and
wherein the one or more apparatus apertures are further configured to fit corresponding one or more illumination components on the distal tip of the endoscope and provide light to the front camera.

52. The smudging mitigation apparatus according to claim 51, wherein the window elements comprise:
a front camera window configured to fit within a central apparatus aperture of the one or more apparatus aperture over the front lens assembly of the front camera, and
one or more illumination component windows configured to fit within one or more surrounding apparatus aperture of the one or more apparatus aperture over the illumination components.

53. The smudging mitigation apparatus according to claim 51, wherein the front end of the distal tip comprises one or more apertures configured to fit the one or more apparatus apertures, the front lens assembly and the illumination components.

54. The smudging mitigation apparatus according to claim 51, wherein the front face comprises mitigating elements, the mitigation elements comprising ridges, rims, bumps and/or grooves, said mitigating elements are configured to prevent fluids, bodily fluids and/or debris from reaching the window elements.

55. The smudging mitigation apparatus according to claim 54, wherein the ridges, rims, bumps and/or grooves are integrally formed with the front face of the smudging mitigation apparatus.

56. The smudging mitigation apparatus according to claim 54, wherein the ridges, rims and/or bumps are attached or mounted to the front face of the smudging mitigation apparatus.

57. The smudging mitigation apparatus according to claim 54, wherein the ridges, rims, bumps and/or grooves are made of plastic, metal, rubber, nylon, or any combination thereof.

58. The smudging mitigation apparatus according to claim 51, wherein the one or more apparatus apertures and the one or more apertures do not hinder the field of view of the front camera.

59. The smudging mitigation apparatus according to claim 51, wherein the diameter of the central apparatus apertures is similar to or larger than the diameter of the front lens assembly and wherein the diameter of the one or more surrounding apparatus apertures is similar to or larger than the diameter of the corresponding illumination components.

60. The smudging mitigation apparatus according to claim 51, wherein the one or more illumination components is or comprises a discrete light source.

61. The smudging mitigation apparatus according to claim 51, wherein a gap is formed between the front end of the distal tip and the back face of the apparatus.

62. The smudging mitigation apparatus according to claim 51, wherein the smudging mitigation apparatus is integrally formed with the front end of a distal tip of the endoscope.

63. The smudging mitigation apparatus according to claim 51, wherein the smudging mitigation apparatus comprises means for fitting and/or securing to the front end of the endoscope tip.

64. The smudging mitigation apparatus according to claim 51, wherein the smudging mitigation apparatus is reusable and sterilizable.

65. The smudging mitigation apparatus according to claim 51, wherein the smudging mitigation apparatus is disposable.

66. The smudging mitigation apparatus according to claim 51, wherein the smudging mitigation is passive.

67. A method for mitigating smudging of a window element positioned on a distal tip of an endoscope, the method comprising:
fitting and securing to a front end of the distal tip of the endoscope a smudging mitigation apparatus having a front face exposed to a body cavity and a back face opposite thereto, said back face is configured to fit on the front end of the distal tip, wherein the smudging mitigation apparatus further comprises one or more apparatus apertures traversing the front face and the back face and configured to correspond to a front lens assembly of a front camera positioned on the front end of the endoscope.

68. The method according to claim 67, wherein the front face of the smudging mitigation apparatus comprises mitigating elements comprising ridges, rims, bumps and/or grooves, the mitigating elements are configured to prevent bodily fluids from reaching the window elements.

69. The method according to claim 67, wherein the method further comprises forming a gap between the front end of the distal tip and the back face of the apparatus.

70. The method according to claim 67, wherein the mitigation of smudging is passive.
